(19) United States
(12) Patent Application Publication
(10) Pub. No.: US 2004/0260154 A1
(43) Pub. Date: Dec. 23, 2004

(54) Human physiological and chemical monitoring system

(76) Inventors: Boris Sidelnik, Aurora, CO (US); Vladimir Oganissian, Aurora, CO (US)

Correspondence Address: SHERIDAN ROSS PC, 1560 BROADWAY, SUITE 1200, DENVER, CO, 80202

(21) Appl. No.: 10/465,704

(22) Filed: Jun. 18, 2003

(51) Int. Cl. A61B 5/00

(52) U.S. Cl. 600/300

(57) ABSTRACT

A human physiological and chemical monitoring system includes a device worn by a person to monitor, detect, record and/or analyze information received from sensors in contact with the wearer's body. Based upon the information generated, the system can indicate that the wearer has likely engaged in a sexual activity.

### FIELD OF THE INVENTION

The present invention relates to systems and methods for monitoring, detecting, recording and/or measuring an individual's sexual activity. It has numerous beneficial and varied applications. For example, it may be utilized as a safety device to monitor the actions of persons in positions of trust or convicted sexual offenders; it may be utilized to prevent the spread of communicable diseases; it may be used by parents or guardians to monitor activities of minors or wards; it may be used as a recording device by vulnerable persons, such as public figures, to combat or defend against wrongful claims of paternity or abuse; it may be utilized for purposes of ascertaining fidelity among spouses or partners; it can be a barrier against undesired or unwelcome attention when worn visibly; and, it may also be utilized as a novelty device to monitor and record responsiveness among consenting partners.

### BACKGROUND OF THE INVENTION

Fidelity in relationships of trust is critical. Recently, sexual abuse of children by persons in a position of trust, such as priests and other religious leaders, has been widely reported. Similarly, trust among consenting partners in an adult relationship is a necessary component to a long-standing relationship. In either scenario, the specific individual makes a voluntary choice to be faithful. Nevertheless, infidelity or abuse still occurs. Sexual abuse of a minor by a person who has the child's trust can devastate a young life. Irrevocable trauma can occur and a lifetime can be spent attempting to overcome the trauma. In the case of consenting partners or spouses, the divorce rate in the United States has risen dramatically over the past thirty years, as has the number of children living with divorced parents. Should these rates continue and marriage become less accepted and more uncommon, the negative impact on society could be significant. Researchers have concluded that when partners enjoy greater security within their relationship, families can be preserved and marriages saved. Research also supports the conclusion that adults who are married do markedly better in virtually every measure of well-being than those who are not married. Married people live longer and are generally more emotionally and physically healthy than unmarried persons. Married persons have lower rates of alcoholism, suicide and mental health problems than unmarried persons. Children who are born to and live with married parents do markedly better in every measure of well-being than children who do not. Research further suggests that sexuality functions best when it is exercised within the confines of marriage.

Another significant issue in society today is the recidivism rate of convicted sexual criminals. While some states have laws requiring registration of these individuals, and even publication of their name and address, some of these individuals choose to ignore registration requirements. Moreover, even if they do follow required procedures and register with local authorities, there is no guarantee that these individuals will not act inappropriately.

Another potential problem area is promiscuity of minors. Pregnancy among teenagers or even younger minors can have lasting negative effects not only on the promiscuous minor, but on their unplanned offspring and other immediate family members who become involved for the sake of the minor and the minor's child. Research confirms that these new parents, and their children, will not financially or emotionally succeed as well as their peers who did not become pregnant as minors.

Public figures and other prominent figures are potential targets of blackmail. Such individuals may be wrongfully subject to paternity claims or claims of sexual misconduct.

Having the ability to monitor and record the person's sexual activity will provide a record for evidentiary purposes to protect against such claims.

In view of the foregoing problems it is an object of the present invention to provide a system and method which is capable of monitoring, detecting and recording sexual activity.

It is another object of the present invention to package such a system into a portable device that would be worn by an individual to measure, on a periodic and/or continuous basis, the sexual activity of that individual. The device may optionally record that information in a manner that can be subsequently retrieved and analyzed or may be capable of internally analyzing acquired information.

It is a further object of the present invention to provide a time and date stamp with each measured activity to, in essence, provide a log of an individual's sexual activity.

Other features could be added to the system to provide enhanced security such as the permanence of the recorded data and the inability of the wearer to access or manipulate the recorded data. In one example, the device could include a transmitter to periodically transfer collected information to a safe, remote site. Further features may be incorporated which would preclude removal of the device or, at least, indicate tampering.

It is yet a further object of the present invention to have persons charged with the trust of minors to wear such a device to thereby prevent abuse of minors.

It is a further object of the present invention to provide a monitor for potentially promiscuous minors. Monitoring and recording the sexual activity of such minors can have a positive preclusive or chilling effect on the minor or a person involved with the minor. The result is avoidance of unwanted pregnancies or sexually transmitted diseases that can occur by abstinence or intimidation to third parties by the presence of the present invention.

Similarly, it is an object of the present invention to provide a system and method of recording sexual activity for evidentiary purposes, which would be available for defending prominent or public individuals against wrongful allegations of misconduct and would similarly be available to prosecute persons of trust or previously convicted sex offenders.

It is a further object of the invention to monitor and record information for consenting adult partners to provide feedback as to responsiveness and intensity.

Other objects and advantages of this invention will become apparent to those of skill in the art from the following description taken in combination with the accompanying drawings.

### SUMMARY OF THE INVENTION

Masters and Johnson, in their well known work in the field of human sexuality, identified four phases of sexual response in males and females. These phases or stages are excitement, plateau, orgasm and resolution. Other authors have identified a fifth and preliminary stage: desire. The stage of desire is considered a prelude to the excitement stage and is understood to occur in the mind, rather than in the body. Therefore, it is difficult to monitor and measure. The excitement stage, on the other hand, is the body's physical response to desire and can be detected and monitored. Besides changes in genitalia, during the excitement stage heart rate and blood pressure increases in both men and women. In addition, skin becomes flushed and breathing accelerates. From a physiological standpoint, the plateau stage is a continuation of the excitement stage. Among other things, in both men and women, breathing rate, heart rate and blood pressure further increase. During the orgasm stage, built up sexual tension is released. From a neurological or chemical standpoint, the neural cells in the brain release endorphins. For example, both men and women produce and release an endorphin or hormone called oxytocin. Men also release testosterone. In addition, heart rate, blood pressure and breathing rate reach their highest peak. At least one author indicates that, on average, the heart rate increases to a maximum of about 120 beats per minute, from a normal of approximately 70 beats per minute. This same author also states that during extramarital sex, the heart rate increases beyond the normal maximum to a heightened maximum of about 180 beats per minute. Resolution is the stage that follows orgasm. Here, for both men and women, heart rate and blood pressure drop below normal levels. In addition, for both sexes, the body sweats. For men, the resolution stage also includes a refractory period during which erection cannot be achieved. The duration of the refractory period varies widely based upon a number of factors, including age.

It is these bodily changes that occur during sexual activity, as well as their interrelationship, that can be monitored, detected, recorded and measured. For example, in one embodiment of the present invention, a person wears a device containing sensors and a miniature processor or controller. The sensors may monitor human physiological activities and/or neurological or chemical activities. Thus, blood pressure, skin temperature, heart rate, breathing rate and/or blood oxygen levels may be monitored from a physiological standpoint. And from a neurological or chemical standpoint, the sensors may monitor for the release of chemicals such as hormones, endorphins and/or sweat. Indeed, the hormones released during sexual activity are likely to be found in sweat. In addition to monitoring these characteristics, the device may include storage capabilities to record these activities, such as recording the information received from the various sensors. In one embodiment, the device may also include a clock to indicate when during a particular time period information was generated and/or recorded. The clock may also be used to interrelate the sensed information for purposes of analyzing the information. In one embodiment, the internal processor or controller of the device maybe capable of analyzing the acquired information. Alternatively, the device may also include a port in order that the stored information can be downloaded to an external computer for analysis of the recorded data and for more permanent storage purposes.

In every embodiment, the device would need a power source of some kind. Preferably, the device would include a rechargeable or replaceable battery of sufficient life or duration to last the full extent of the intended monitoring period. In another embodiment, one of the ports could be used to recharge the power supply to provide power during use, if a power source is available and convenient.

In another embodiment, the device could include a transmitter for purposes of transmitting the acquired information to a remote and safe location.

### BRIEF DESCRIPTION OF THE DRAWINGS

FIG. 1 is a block diagram of one embodiment of the system of the present invention.

FIG. 2 is a perspective view of one embodiment of a device for monitoring human physiological and/or neurological/chemical characteristics of the human body.

FIG. 3 is a cross-sectional view of the embodiment depicted in FIG. 2.

It should be understood that the drawings are not necessarily to scale. In certain instances, details which are not necessary for an understanding of the invention or which render other details difficult to perceive may have been omitted. It should be understood, of course, that the invention is not necessarily limited to the particular embodiments illustrated herein.

### DETAILED DESCRIPTION OF THE PREFERRED EMBODIMENT

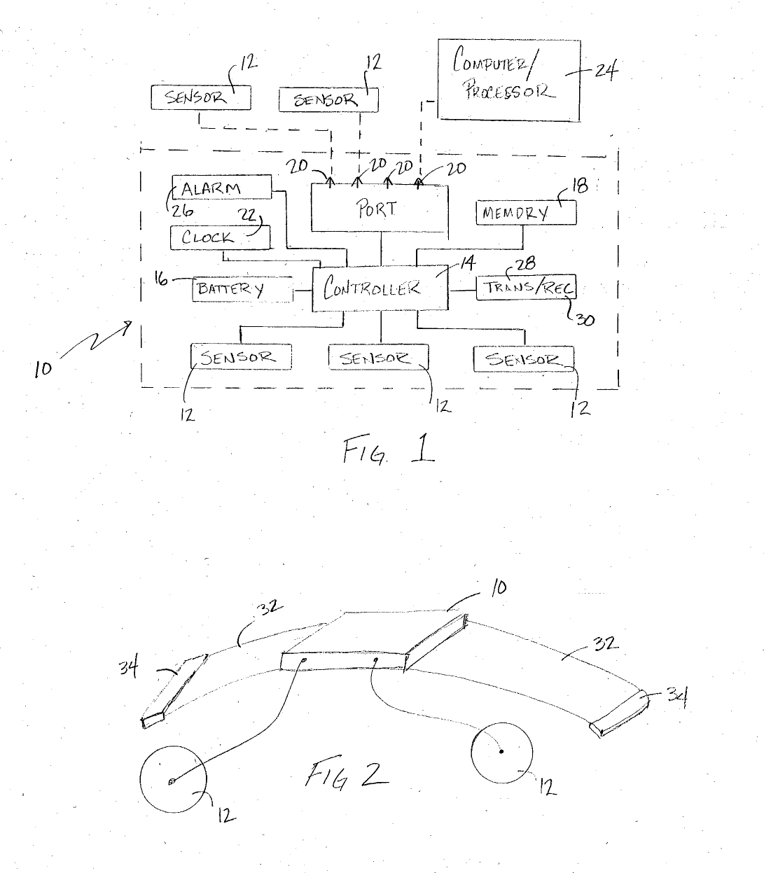

Turning to FIG. 1, a block diagram of one embodiment of the present invention is shown. As can be seen the monitoring device 10 has a plurality of sensors 12. The number and type of sensors may vary, as will be understood by those skilled in the art. The sensors may monitor physiological characteristics which directly or indirectly indicate sexual activity, such as temperature, blood pressure, heart rate, breathing rate, blood oxygen and/or blood volume or flow. The sensors may also monitor chemical or neurological characteristics which directly or indirectly indicate sexual activity. For example, during sexual activity the body will release or secrete hormones such as endorphins, including oxytocin and/or testosterone, and it is believed that these hormones are a constituent component of perspiration or sweat. As should be appreciated, the sensors may monitor any combination of physiological and/or chemical characteristics.

The monitoring device of the preferred embodiment further includes a controller or processor 14, a power source or battery 16, a memory or storage area 18, and at least one port 20. The controller controls and coordinates the various components of the device. For example, the controller may contain software or firmware that instructs the sensors to take readings at predetermined time intervals. It may change or alter the timing of the intervals based upon information acquired from or feedback from the sensors. For example, should one or more monitored characteristics change by a particular amount or percentage, the controller may instruct the sensors to take more frequent or less frequent readings. Alternatively, the controller could instruct the sensors to take continuous readings or to stop taking readings, for example, if the monitored characteristics move outside of a predetermined range. The frequency of readings can vary as needed and based upon the characteristic being monitored.

The memory 18 stores the information or data collected by the sensors. One example of an acceptable memory device would be an electronic erasable programmable read only memory (EEPROM). Such a device is small enough to be relatively unobtrusive when assembled into a monitoring device such as this and is erasable and rerecordable allowing it to be reused. Other examples include erasable programmable read only memory (EPROM) or static random access memory (SRAM). The latter memory type device requires a power source to maintain its memory capabilities. Still further examples of suitable memory devices will occur to those skilled in the art of miniaturized recording techniques upon review of this disclosure.

As should be appreciated, for the device to be portable, a portable power source is also required. The battery 16 provides power to the various components. The battery may be rechargeable or replaceable. The size and/or strength of the battery would be selected to meet the time duration requirements of the device, as well as the consumption based upon the number of sensors utilized and other operations being performed, for example, powering the controller, memory and other components.

The device of the preferred embodiment would also include a clock 22. The clock would provide a time mark as to when detected activities occurred. It would also allow the information from the individual sensors to be synchronized for analysis purposes.

The device may optionally include multiple ports 20. The ports may serve a variety of purposes. In one example, one port could be used to upload the stored data to a remote or independent and more powerful computer 24. The computer could contain software for analyzing, formatting, cataloging and storing the collected information. The external computer would also permit printing of reports containing all or some of the collected data in desired a format. The ports may also be used to connect auxiliary components or peripheral devices, such as additional sensors. Another example of a peripheral device would be a printer to directly print information from memory 18, rather than using an external computer. A port could also be used to recharge the internal power supply 16.

In a second embodiment of the present invention, the device 10 could include all of the necessary software or firmware to analyze the collected data, rather than using an external computer for this purpose. The controller or processor 14 would not only control information acquisition from the sensors, but analyze the acquired information as well. A necessary requirement of such a system would be adequate battery life to sustain monitoring, data collection and data analysis over sustained time periods. Of course, an extended power supply (not shown) could be connected to the device 10 via one of the ports during periods of high power usage, such as during analysis of data. The processor could also perform many of the functions identified as separate components, including but not limited to the clock function, the memory function and other functions known to those of ordinary skill in the art.

In a third embodiment, the device could include an alarm 26 intended to activate if the monitored data suggests sexual activity is occurring. The alarm could be audible to intimidate or frighten either the person wearing the device or another person. Alternatively, the alarm could be audibly silent but perceptible to the wearer nonetheless, such as a mild shock applied to the person wearing the device. The alarm could intensify if the activity continues or the monitored characteristics intensify. The intent is to prevent a sexual act from occurring. Other forms of alarms or warnings within the scope of the present invention will occur to those of skill in the art upon reading this disclosure.

In a fourth embodiment of the present invention, the device could include a transmitter 28 for transferring collected data to a remote device. This would permit transfer of data to prevent loss of data and/or to permit real-time or near real-time monitoring by third parties and remote locations. The transmitter could operate using wireless techniques, such as by radio frequency technology, including inductive coupling with a nearby active device or, if adequate power is available, the device could transmit directly to a remote receiver. Infrared transmission is another possibility, but would require an unimpeded line of sight between the transmitter and receiver. The device could also include a receiver 30 to receive commands from an external device. Such wireless communications can be used to modify existing software resident in the device rather than using a physical, hard-wire connection to an external device. Thus, instead of having the person wearing the device report to a fixed-location for uploading the collected data to an external computer or storage device and reprogramming of the device 10, a portable programming device could be taken to the location of the person wearing the monitoring device 10 for purposes of acquiring stored data, checking system operations and/or altering programming of the device. This would also permit surprise or unexpected visits by oversight personnel, further enhancing at least one objective of the overall system, namely, preventing sexual activity.

As a further alternative, the transmitter 28 could be a global positioning transmitter in order that the location of the person wearing the device can be tracked. This capability would be particularly useful if the device 10 was capable of transmitting sensed information in real-time or near real-time to a remote monitoring terminal where observers could directly monitor the wearer's activities. This would permit oversight personnel to intervene if the data indicated sexual activity was occurring.

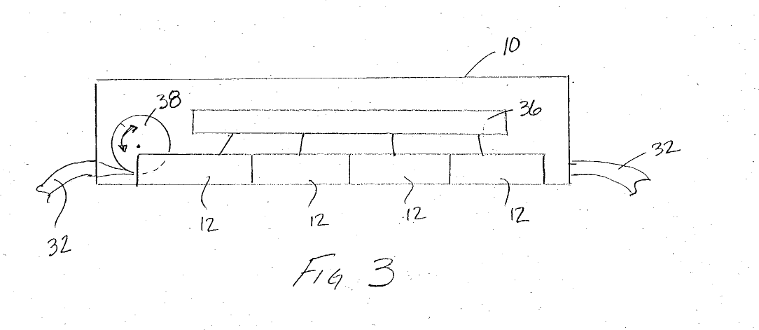

FIG. 2 illustrates one physical embodiment of the present invention. The monitoring device 10 is attached to a strap 32 allowing it to be worn around one or more parts of the human body. Continuous contact with the skin of the person wearing it is important in order to obtain uninterrupted monitoring and collection of data. The strap further includes a securement mechanism 34 permitting the strap to be attached and detached from the wearer. Preferably, the securement mechanism is tamper proof so that it can only be removed by an authorized person or, at a minimum, it will indisputably show that it has been tampered with.

In yet another embodiment, the sensors 12 could be external to the monitoring device 10 as shown in FIG. 2. For example, one or more of the sensors could be in the form of skin patches, such as are used to monitor sweat gland secretions. These sensor patches could be hard wired to the monitoring device 10 via ports 20. While such a system is more susceptible to tampering, not all wearers of the monitoring device will be hostile to wearing it. In any event, the processor or controller can be adapted to detect interruption of data collection, such as would occur if the device was removed or the ability of any sensors to collect data was altered. The duration of the interrupted functioning can be recorded in the memory 18 and the device could also be programmed for the alarm to sound, or for a signal to be transmitted to an external wireless receiver at a remote monitoring station upon detection of possible tampering.

In operation, the monitoring device can monitor physiological and/or chemical characteristics of the human body. For example, it can monitor, measure and record heart rate, blood pressure, temperature and/or breathing rate, as well as the secretion of hormones, such as endorphins, and sweat (which would likely include the desired hormones). Importantly, it can also apply a time line to these monitored characteristics such that the interrelationship of these characteristics can be analyzed and sexual activity detected. For example, should blood pressure, heart rate and breathing rate increase over a period of time, with the heart rate reaching 120 beats per minute or greater, followed by a period of below normal heart rate, this data would indicate sexual activity. Alternatively, should this pattern of physiological characteristics occur together with the secretion of endorphins, or should the wearer secrete sweat following the described pattern of increased heart rate, blood pressure, temperature and breathing rate, it would also confirm sexual activity.

A key to detecting sexual activity is by its unique pattern of bodily characteristics. The pattern or interrelationship of various physiological and/or chemical characteristics of the human body may be used to distinguish sexual activities from other activities, such as physical exercise. It is believed that the interrelationship of some or all of these physiological and/or chemical characteristics creates a unique finger print of the occurrence of sexual activity and, thus, may be accurately monitored. It is also believed that monitoring and analyzing other physiological characteristics and/or other chemical secretions or neurological characteristics, as would occur to those of skill in the art upon reading the present disclosures, would also accurately indicate sexual activity. Monitoring and analyzing such additional characteristics and secretions are deemed to fall within the scope of the present invention.

It is believed that appropriate technology exists today to construct an operable and commercially feasible device. For example, for monitoring heart rate, the sensor could be analogous to a cardiac monitor. To perform optimally, a pair of electrodes are positioned on the chest wall of the wearer, similar to that of an electrocardiograph. The electrodes would be external to the main housing of the device, such as sensors 12 in FIG. 2, or one or both of the electrodes could be the sensors shown in FIG. 3. In addition, an amplifier (not shown) is preferably included to strengthen the signals from the electrodes. The amplifier could be added to the printed circuit board 36, which contains the other electronic components identified in FIG. 1. Such an amplifier is preferably a biopotential amplifier which is designed to take a weak electric signal of biological origin, such as the heart, and increase its amplitude. It is usually in the form of a voltage amplifier.

Blood pressure may be monitored indirectly using an occlusive cuff and a distal monitoring device. This technique is well known. In connection with the present invention, as shown in FIG. 3, the strap 32 may be designed to act as an occlusion device. In one embodiment, the strap may be inflatable and a miniaturized pump 38 included within the housing, and under the control of the controller 14. The pump would inflate the strap, creating an occlusion and one or more of the sensors could monitor blood pressure. Alternatively, in a second embodiment, the pump would be replaced by a miniaturized winch or similar winding-type device which could tighten the strap for periods of short duration, creating an occlusion and permitting blood pressure measurement. In a further embodiment, blood pressure may be monitored ultrasonically. This technique would utilize a transcutaneous doppler sensor that detects motion of blood vessel walls. It also requires an occlusion cuff. Another possible technique known to those of skill in the art is the oscillometric method, which requires an occlusion cuff.

It is believed that acceptable techniques also exist for monitoring breathing rates. One example is electric-impedance plethysmography which is used to measure changes in tissue volume and, when electrodes are placed on each side of the thorax, this technique provides a good indication of rate of ventilation, but a less accurate indicator of volume of ventilation. This technique is used for monitoring infant apnea. In one embodiment of the present application, external sensors 12 as shown in FIG. 2, may be applied to opposite sides of the thorax for monitoring breathing rate.

Blood oxygen level is believed to be related to breathing rate. Oxygen levels may rise with an increased breathing rate or increased physical activity. Similarly, blood flow or volume is believed to be related to heart rate. Therefore, monitoring blood oxygen levels and/or blood volume or flow may be another indicator of sexual activity, particularly in combination with one or more of the other characteristics. Photoplethysmographic techniques may be used to monitor oxygen content in the blood and/or blood flow or volume. In theory, as arterial pulsations fill capillaries, the changes in the volume of blood in the capillaries modify the absorption, reflection and scattering of light. In one technique, light is transmitted into a finger where it reflects off bone and detected by a photosensor. Alternatively, the light maybe transmitted through an ear or similar thin body part. A silicon photo transistor is an example of a photosensor. A miniature tungsten lamp may serve as the light source. Other techniques would be known to those of skill in the art of the measurement of flow and volume of blood.

Thermal sensors may be used to monitor temperature. A thermal sensor absorbs radiation and transforms it into heat, thus causing a rise in temperature in the sensor. Known thermal sensors include thermal couples and thermistors. A pyroelectric sensor may also be acceptable.

It is also believed that existing technology will allow for chemical sensing of secreted hormones and sweat. It is believed that sweat may be monitored in a manner similar to the operation of a pH sensor. Measuring pH can be accomplished using a glass electrode that generates an electric potential when solutions of differing pH are placed on two sides of a membrane. A solution of a known pH is stored on one side, and the skin of the wearer is placed in contact with the other side. It is believed that sweating changes the pH of the skin and, thus, the differential can be detected. Similarly, the detection of hormones can be accomplished in the same fashion. A solution of known composition, containing normal levels of hormones, would be stored for comparison to chemicals secreted by the wearer's sweat glands. Comparison of the two would create a differential during sexual activity or shortly thereafter. This differential can be designed to create an electric potential which can be detected and measured. An amplifier can be incorporated to magnify the amplitude of the signal.

The foregoing discussion of the invention has been presented for purposes of illustration and description. The foregoing is not intended to limit the invention to the form or forms disclosed herein. In the foregoing description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this description, with each claim standing on its own as a separate preferred embodiment of the invention.

Moreover, though the description of the invention has included description of one or more embodiments and certain variations and modifications, other variations and modifications are within the scope of the invention, e.g. as may be within the skill and knowledge of those in the art, after understanding the present disclosure. Indeed, the present invention involves numerous fields or arts and variations or modifications known to those of skill in the involved areas are deemed to be within the scope of this invention. The present invention is intended to cover rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for monitoring and detecting sexual activity by an individual human being using a portable device, comprising:
a. monitoring one or more physiological characteristics of the human body including at least one of blood pressure, heart rate, breathing rate, blood oxygen levels and temperature;
b. monitoring one or more chemical characteristics of the human body including monitoring for the presence of at least one of hormones or sweat;
c. analyzing the monitored characteristics;
d. determining if a sexual activity likely occurred.

2. The method of claim 1, further comprising recording one or more of the monitored characteristics.

3. The method of claim 1, further comprising transferring the recorded data to a second device.

4. The method of claim 3, further comprising using the second device to analyze the recorded data.

5. The method of claim 1, further comprising transferring the monitored data to a second device.

6. The method of claim 5, further comprising recording the transferred data at the second device.

7. The method of claim 5, further comprising transferring the monitored data to a second device using wireless technology.

8. The method of claim 1, further comprising communicating with a remote location that a sexual activity has likely occurred.

9. The method of claim 1, further comprising attaching the monitoring device to a person in a secure manner.

10. The method of claim 9, further comprising attaching the device to a person in a manner which indicates if it is tampered with.

11. The method of claim 1, further comprising determining the intensity of the likely sexual activity based upon the monitored activity.

12. A method of monitoring a person's sexual activity, comprising:
a. attaching at least one portable device to the body of the person;
b. sensing a plurality of human characteristics indicative of sexual activity;
c. generating information based upon the sensed characteristics;
d. determining if sexual activity likely occurred based upon the information.

13. The method of claim 12, wherein the sensed human characteristics include secretions of sweat glands.

14. The method of claim 13, wherein the sensed human characteristics include chemicals found in sweat.

15. The method of claim 14, wherein the sensed chemicals comprise at least one of hormones, pheromones, endorphins, oxytocin or testosterone.

16. The method of claim 12, wherein the sensed human characteristics are physiological characteristics.

17. The method of claim 16, wherein the sensed physiological characteristic is at least one of blood pressure, temperature, heart rate, breathing rate or blood oxygen levels.

18. The method of claim 12, wherein the sensed human characteristics include at least one physiological characteristic and at least one chemical secreted by sweat glands.

19. The method of claim 12, further comprising recording information developed by the sensors.

20. The method of claim 19, further comprising recording each sensed characteristic at a plurality of different times.

21. The method of claim 19, further comprising analyzing the recorded information.

22. The method of claim 21, wherein analyzing the recorded information comprises transferring the information from the device to a remote device.

23. The method of claim 21, further comprising indicating if a sexual activity is likely occurring.

24. The method of claim 23, wherein the indication is an audible sound.

25. The method of claim 23, wherein the indication is a non-audible sensation felt by the person wearing the device.

26. The method of claim 23, wherein the indication is recorded.

27. The method of claim 12, wherein the sensing occurs over a time period

28. The method of claim 27, wherein the sensing occurs continuously over a time period.

29. The method of claim 27, wherein the sensing occurs periodically over a time period.

30. The method of claim 29, wherein information generated by sensing is recorded based upon variation of the values of the sensed information.

31. A system for monitoring and detecting human sexual activity, the system comprising:
a. a portable device worn by a person, the portable device comprising:
i. a plurality of sensors positioned in contact with the body of a person, each sensor monitoring a human physiological characteristic or body secretion;
ii. a storage device for recording information generated by the sensors;
iii. a controller for controlling the sensors, the information generated by the sensors and the storage device;
iv. a second device, remote from the person, the second device comprising a processor for analyzing the information maintained in the storage device and for determining if a sexual activity likely occurred.

32. The system of claim 31, wherein the physiological characteristics include at least one of blood pressure, temperature, breathing rate, blood oxygen levels and heart rate.

33. The system of claim 31, wherein the body secretion includes at least one of hormones, pheromones, oxytocin or testosterone.

34. The system of claim 31, further comprising means for the portable device to communicate with the second device.

35. The system of claim 34, wherein the communication means is wireless.

36. The system of claim 31, further comprising an attachment mechanism to secure the portable device to the body of the person whereby it cannot be readily removed without authorization.

37. The system of claim 31, wherein at least one of the sensors is a patch worn on the skin of the person.

38. An apparatus for monitoring human sexual activity, comprising:
a. a plurality of sensors adapted to be worn in contact with the body of a person and for generating information;
b. a controller connected to the sensors for controlling some actions of the sensors;
c. a power supply in communication with the controller and one or more of the sensors; and
d. a storage device for storing information generated by the sensors.

39. The apparatus of claim 38, wherein the sensors, controller, power supply and storage device are disposed in a single housing.

40. The apparatus of claim 38, further comprising at least one port for communicating with a second device.

41. The apparatus of claim 40, wherein the second device is a sensor affixed to the body of a person.

42. The apparatus of claim 40, wherein the second device is an external processor for processing information.

43. The apparatus of claim 38, wherein each sensor monitors a physiological characteristic or a chemical secretion of a human body.
